(19) United States
(12) Patent Application Publication
(10) Pub. No.: US 2021/0339041 A1
(43) Pub. Date: Nov. 4, 2021

(54) LIGHT THERAPY DEVICE HAVING LIGHT DIFFUSING FUNCTION

(71) Applicant: CHEOMDANLAB INC., Gwangju (KR)

(72) Inventors: Ha Jun JANG, Gwangju (KR); Ji Yun JEONG, Gwangju (KR); In Ha SEO, Jeonju-si (KR); Yeol Ho LEE, Gwangju (KR); Hyun JEONG, Gwangju (KR); Seok Jae KIM, Gwangju (KR); Ju Won JEONG, Gwangju (KR)

(73) Assignee: CHEOMDANLAB INC., Gwangju (KR)

(21) Appl. No.: 17/057,733

(22) PCT Filed: Aug. 3, 2020
(86) PCT No.: PCT/KR2020/010206

§ 371 Date: Nov. 23, 2020

(30) Foreign Application Priority Data
May 4, 2020 (KR) 10-2020-0053385

(51) Int. Cl. A61N 5/06; G02B 5/02

(52) U.S. Cl. CPC A61N 5/0616; G02B 5/0247; A61N 2005/0651; A61N 2005/0661; A61N 2005/0662; G02B 5/0278

(57) ABSTRACT

Provided is a light therapy device capable of further maximizing a scattering effect, having enhanced durability, and exhibiting energy uniformity sufficient for use as a light therapy device.

### TECHNICAL FIELD

Disclosed embodiments relate to a light therapy device having a light diffusing function.

### BACKGROUND ART

Recently, light therapy devices for performing skin therapy using a plurality of light-emitting diode (LED) elements have been widely used.

In general, the LED elements are solid light-emitting elements and are known to have very high luminous efficiency. However, when used for the light therapy devices, light uniformity is required to increase a therapy effect, and thus a greater number of LED elements are needed to be used.

When a great number of LED elements are used, problems such as power consumption, heat generation, or the like may occur.

### DESCRIPTION OF EMBODIMENTS

### Technical Problem

In order to solve the limitations of light therapy devices as described above, according to an embodiment, a light therapy device capable of improving light uniformity and solving power consumption and heat generation problems may be provided.

According to another embodiment, a light therapy device having a further improved therapy effect may be provided.

### Solution to Problem

To achieve the objects as described above, according to an embodiment of the disclosure, a light therapy device includes a first support positioned adjacent to skin, the first support being transmissive, a second support positioned opposite to the first support, the second support being non-transmissive, and a flexible light-emitting assembly positioned between the first support and the second support, the flexible light-emitting assembly including a light diffuser and a light-emitting element, wherein the light diffuser includes a base material including a first surface and a second surface, which face each other, the base material being configured to allow light from the light-emitting element to be incident on the first surface and emitted out from the second surface, wherein the base material is configured to scatter the light when the light passes therethrough.

The base material may include a plurality of pores irregularly distributed in the base material, and the scattering may include first scattering due to the pores and second scattering due to at least one of the first surface and the second surface.

The first scattering degree due to the first scattering and the second scattering degree due to the second scattering may have a relative difference.

When the first scattering degree due to the first scattering is greater than the second scattering degree due to the second scattering, each of the pores may have a first diameter, and when the second scattering degree due to the second scattering is greater than the first scattering degree due to the first scattering, each of the pores may have a second diameter, wherein the first diameter may be greater than the second diameter.

A roughness of at least one of the first surface and the second surface when the first scattering degree due to the first scattering is greater than the second scattering degree due to the second scattering, is a first roughness, and a roughness of at least one of the first surface and the second surface when the second scattering degree due to the second scattering is greater than the first scattering degree due to the first scattering, at least one of the first surface and the second surface, is a second roughness, wherein the first roughness may be less than the second roughness.

The light therapy device may further include a molding support positioned between the first support and the second support.

The light therapy device may further include a bonding support positioned between the skin and the light diffuser, the bonding support being bondable to the skin.

### Advantageous Effects of Disclosure

According to an embodiment of the disclosure as described above, by arranging a flexible light diffuser on an entire surface of a light path of a light-emitting element, light having straightness may be sufficiently diffused, and accordingly, light energy uniformity sufficient for use as a light therapy device may be provided.

The flexible light diffuser may transfer energy of each wavelength of the light-emitting element as it is without any changes in a spectrum from an ultraviolet (UV) light region to a visible light region. That is, light transmitted through the light diffuser may be transferred to an irradiation surface while minimizing loss in the light power. According to an embodiment, the light diffuser does not cause a shift in wavelengths, and a wavelength band of the light transmitted may be maintained as it is.

Also, because the light diffuser may uniformly form an in-plane light source, light energy may be uniformly transferred over an entire surface.

Moreover, because the light diffuser is flexible, uniform light characteristics do not change even though the light diffuser is applied to uneven skin. Also, due to a diffusing function, a micro element may be used as the light-emitting element, and even when a micro light-emitting element is used, a sufficient in-plane light source can be achieved, so that the number of light-emitting elements may be reduced. When the number of light-emitting elements is reduced as described above, a heat generation problem caused due to a great number of light-emitting elements may be solved.

### BRIEF DESCRIPTION OF DRAWINGS

FIG. 1 is a schematic cross-sectional view of a flexible light-emitting assembly according to an embodiment.

FIG. 2 is an enlarged cross-sectional view of an area A of FIG. 1.

FIG. 3 is a partial enlarged cross-sectional view of a portion of FIG. 1.

(a) of FIG. 4 is a cross-sectional scanning electron microscope (SEM) photograph of an embodiment, and (b) of FIG. 4 is a surface SEM photograph of an embodiment.

(a) of FIG. 5 is a cross-sectional SEM photograph of Comparative Example, and (b) of FIG. 5 is a surface SEM photograph of Comparative Example.

FIGS. 6A to 6D are cross-sectional views of operations for manufacturing a flexible light-emitting assembly according to an embodiment.

FIG. 7 is a schematic cross-sectional view of a flexible light-emitting assembly according to another embodiment.

FIG. 8 is a cross-sectional view of a portion of a light therapy device according to an embodiment.

FIG. 9 is a block diagram of a light therapy device according to another embodiment.

FIG. 10 is a cross-sectional view of a light therapy device taken along a line B-B of FIG. 9.

### MODE OF DISCLOSURE

Because the disclosure may have diverse modified embodiments, particular embodiments are illustrated in the drawings and are described in the detailed description. The effects and features of the disclosure and the accompanying methods thereof will become apparent from the following description of the embodiments, taken in conjunction with the accompanying drawings. However, the disclosure is not limited to the embodiments described below and may be embodied in various modes.

The present embodiments will now be described more fully with reference to the accompanying drawings. When describing embodiments with reference to the accompanying drawings, the same or corresponding elements are denoted by the same reference numerals and a redundant description thereof will be omitted.

As used herein, the singular forms “a,” “an,” and “the” are intended to include the plural forms as well, unless the context clearly indicates otherwise.

It will be understood that terms such as “comprise,” “include,” and “have” used herein specify the presence of stated features or elements, but do not preclude the presence or addition of one or more other features or elements.

It will be understood that when a layer, region, or element is referred to as being “on” another layer, region, or element, it may be directly on the other layer, region, or element or may be indirectly on the other layer, region, or element with intervening layers, regions, or elements therebetween.

When a certain embodiment may be implemented differently, a specific process order may be performed differently from the described order. For example, two processes described in succession may be performed substantially simultaneously or may be performed in an order opposite to that described.

Sizes of elements in the drawings may be exaggerated or contracted for convenience of explanation. For example, because sizes and thicknesses of elements in the drawings are arbitrarily illustrated for convenience of explanation, the following embodiments are not limited thereto.

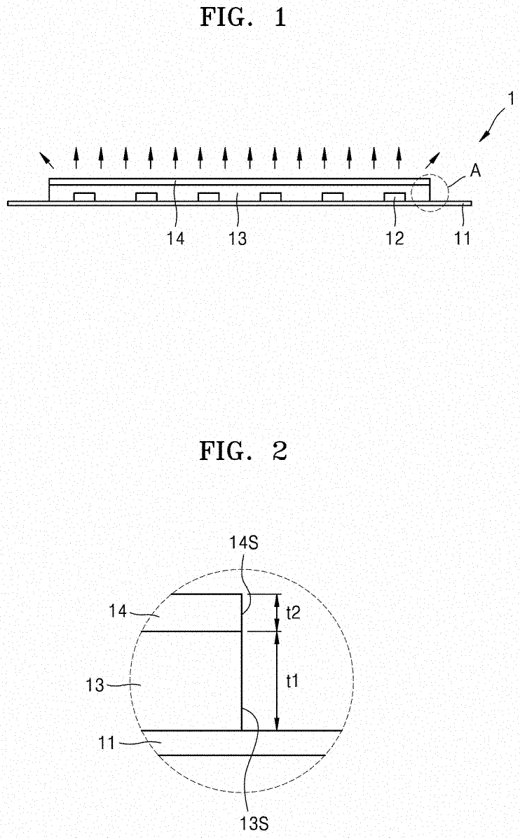

FIG. 1 is a schematic cross-sectional view of a flexible light-emitting assembly 1 according to an embodiment.

The flexible light-emitting assembly 1 according to an embodiment may include a flexible substrate 11, a plurality of light-emitting elements 12, a first film 13, and a second film 14.

A flexible printed circuit board (FPCB) may be used as the flexible substrate 11, and a connecting terminal (not shown) may be connected to one end of the flexible substrate 11. Although not shown, a circuit wire may be patterned on the flexible substrate 11, and elements (not shown) electrically connected to the circuit wire may be further provided on one side of the flexible substrate 11.

The light-emitting elements 12 may be coupled to one surface of the flexible substrate 11. The light-emitting elements 12 may be light-emitting diode (LED) modules, but the disclosure is not limited thereto. Various light-emitting elements such as organic light-emitting diodes (OLEDs), field emission displays (FEDs), etc. may be used.

A wavelength implemented by the light-emitting elements 12 may be included in a first wavelength band of about 405 nm to about 420 nm. The first wavelength band of light may be absorbed into epithelial tissues of the skin and stimulate porphyrins to produce more single oxygen within cells. Accordingly, the first wavelength band of light may be used to destroy bacteria of the epithelial tissues and is thus useful for the treatment of acne or the like.

In another embodiment, the wavelength band implemented by the light-emitting elements 12 may include a second wavelength band of about 630 nm to about 640 nm. The second wavelength band of light may penetrate down to dermal tissues of the skin, and about 80% of light energy thereof may be absorbed into the skin by 2 cm or less, thereby stimulating mitochondria and activating adenosine triphosphate (ATP) production. This may induce cellular turnover, superficial circulation and/or anti-inflammatory emission.

Moreover, in another embodiment, the wavelength band implemented by the light-emitting elements 12 may include a third wavelength band of about 800 nm to about 900 nm. Light in the third wavelength band may penetrate deeper into the skin than visible light, and 50% of the light may penetrate into the skin up to 8 cm. Regarding cells that have absorbed the light in the third wavelength band, the temperature thereof is increased and a pain relief effect may be induced.

The light-emitting elements 12 may be elements emitting light having a single wavelength. According to an embodiment, the light-emitting elements 12 may include a light-emitting element emitting light having at least one of the first to third wavelength bands.

However, embodiments are not limited thereto, and a multi-wavelength device assembly in which a plurality of adjacent light-emitting elements of the light-emitting elements 12 emit light having different wavelengths, may be implemented. For example, a first light-emitting element emits the first wavelength band of light, a second light-emitting element, adjacent to the first light-emitting element, emits the second wavelength band of light, and a third light-emitting element, adjacent to the first light-emitting element and/or the second light-emitting element, emits the third wavelength band of light. The first to third light-emitting elements constitute a set, and a plurality of sets of the first to third light-emitting elements may be arranged. In this case, various light therapy effects may be obtained by using a single light therapy device.

The first film 13 may be attached to one surface of the flexible substrate 11. The first film 13 is attached to one surface of the flexible substrate 11 and may be formed to cover all of the plurality of light-emitting elements 12 formed on the flexible substrate 11.

The first film 13 may include a transmissive material. According to an embodiment, the light-emitting elements 12 may be sealed by the first film 13. The first film 13 may selectively include a waterproof/moistureproof film. Accordingly, the light-emitting elements 12 may have a waterproof function due to the first film 13.

The second film 14 may be positioned on one surface of the first film 13. The second film 14 is attached onto the first film 13. In an embodiment, the second film 14 may be bonded to the first film 13. The second film 14 may include a transmissive material.

As shown in FIG. 2, a first side wall 13S may be provided on one side of the first film 13, and a second side wall 14S may also be provided on one side of the second film 14, which is positioned at the same side as the one side of the first film 13. According to an embodiment, the first side wall 13S and the second side wall 14S may form a continuous surface connected to each other. Accordingly, when assembling the flexible light-emitting assembly 1 into the light therapy device to be described below, assembly consistency with other components may be increased, and a design margin may be obtained.

The first film 13 may have a first thickness t1, and the second film 14 may have a second thickness t2. According to an embodiment, the first thickness t1 may be greater than the second thickness t2. Accordingly, even though each light-emitting element 12 has a certain thickness, the light-emitting elements 12 may be sufficiently covered by the first film 13. Also, because the thickness of the second film 14 is small, an entire thickness of the first film 13 and the second film 14 may be reduced.

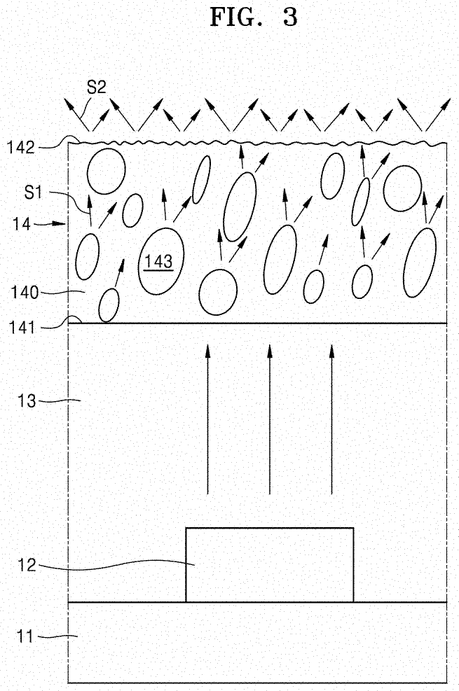

In the flexible light-emitting assembly 1 as described above, according to an embodiment, the second film 14 may be provided as a light diffuser as shown in FIG. 3.

Referring to FIG. 3, the second film 14 being the light diffuser may include a base material 140 and a plurality of pores 143.

The base material 140 may include a transmissive polymer material. According to an embodiment, the base material 140 may include polyimide. The base material 140 may be flexible.

The base material 140 includes a first surface 141 and a second surface 142 which face each other. In this regard, the first surface 141 may serve as an incident surface on which light emitting from the light-emitting elements 12 is incident, and the second surface 142 may serve as an exit surface from which light is emitted. Therefore, light may enter the base material 140 through the first surface 141 and leave the same through the second surface 142.

As shown in FIG. 3, the base material 140 may include the plurality of pores 143 which are irregularly distributed between the first surface 141 and the second surface 142. The pores 143 may function as light scattering particles, form a hollow cavity, and have a refractive index of air in the hollow cavity. In FIG. 3, it is shown that the pores 143 have an elliptical shape. However, embodiments are not limited thereto, and the pores 143 may have various shapes. Also, it is shown that the pores 143 are spaced apart from each other with a space therebetween, but embodiments are not limited thereto. The pores 143 may be in close contact with each other in at least some areas.

The base material 140 as described above may serve to scatter light when the light passes therethrough.

Such scattering may include first scattering S1 due to the pores 143 and second scattering S2 due to at least one of the first surface 141 and the second surface 142.

The light passing through the base material 140 hits the pores 143 irregularly arranged on its path and is thus scattered due to a difference in refractive index between air forming the pores 143 and a polymer constituting the base material 140. The first scattering S1 may include Mie scattering. The first scattering S1 may be scattering of light in which most light spreads in a traveling direction of the light.

The light passing through the base material 140 may be subjected to a second scattering S2 due to at least one of the first surface 141, which is the incident surface, and the second surface 142, which is the exit surface. According to an embodiment, the second scattering S2 may include scattering due to the second surface 142. The second scattering S2 may include surface scattering. In the second scattering S2, scattered light may spread not only in a traveling direction of the light, but also in directions other than the traveling direction, and may also spread in a lateral direction.

In an embodiment, the light diffuser 14 may have a first scattering degree due to the first scattering S1 and a second scattering degree due to the second scattering S2. In this regard, the first scattering degree may be greater than the second scattering degree.

According to an embodiment, in the light diffuser 14, when the first scattering degree due to the first scattering S1 is greater than the second scattering degree of the second scattering S2, an average total transmittance of the base material 140 to a wavelength of the light may be 70% or higher. In this regard, an average total reflectance of the base material 140 to the wavelength of the light may be less than 20%. The average total transmittance with respect to the wavelength of the light may correspond to an average value of a total integral transmittance exhibited when a wavelength of light changes. The average total reflectance with respect of the wavelength of the light may correspond to an average value of a total integral reflectance exhibited when the wavelength of the light changes.

In the light diffuser 14, when the first scattering degree due to the first scattering S1 is greater than the second scattering degree due to the second scattering S2, the light diffuser 14 may have high transparency and low reflectivity. Moreover, in this case, an average light diffusion (haze) value with respect to the wavelength of the light may be about 80% or higher. Also, when the light diffuser 14 is attached to the light therapy device, light extraction efficiency of the light therapy device may be improved, and high power efficiency of the light therapy device may be achieved.

When the first scattering degree due to the first scattering S1 is greater than the second scattering degree due to the second scattering S2, a light diffusion (haze) value of the light diffuser 14 may decrease to a first angle as the wavelength of the light increases. When the second scattering degree due to the second scattering S2 is greater than the first scattering degree due to the first scattering S1, the light diffusion (haze) value of the light diffuser 14 may decrease to a second angle as the wavelength of the light increases. In this regard, the second angle may be greater than the first angle. Accordingly, the light diffuser 14 in a case where the first scattering degree due to the first scattering S1 is greater than second scattering degree due to the second scattering S2 has a higher average light diffusion (haze) value according to the wavelength of the light, compared to the light diffuser 14 in a case where the second scattering degree due to the second scattering S2 is greater than the first scattering degree due to the first scattering S1. That is, in terms of light diffusion, the light diffuser 14 in a case where the first scattering degree due to the first scattering S1 is greater than second scattering degree due to the second scattering S2 may exhibit relatively excellent characteristics, compared to the light diffuser 14 in a case where the second scattering degree due to the second scattering S2 is greater than the first scattering degree due to the first scattering S1.

In the light diffuser 14 according to an embodiment, when the first scattering degree due to the first scattering S1 is greater than the second scattering degree due to the second scattering S2, each pore 143 has a first diameter, and when the second scattering degree due to the second scattering S2 is greater than the first scattering degree due to the first scattering S1, each pore 143 has a second diameter. In this case, the first diameter may be greater than the second diameter.

Optionally, when the first scattering degree due to the first scattering S1 is greater than the second scattering degree due to the second scattering S2, a surface roughness of at least one of the first surface 141 and the second surface 142 may be a first roughness, and when the second scattering degree due to the second scattering S2 is greater than the first scattering degree due to the first scattering S1, a surface roughness of at least one of the first surface 141 and the second surface 142 may be a second roughness. In this regard, the first roughness may be less than the second roughness.

That is, in the light diffuser 14, it is preferable that a size of each pore 143 is greater and the surface roughness of at least one of the first surface 141 and the second surface 142 is less.

When the first scattering degree due to the first scattering S1 is greater than the second scattering degree due to the second scattering S2, the size of each pore 143 may have a more significant effect on the first scattering S1. According to an embodiment, when the first scattering degree due to the first scattering S1 is greater than the second scattering degree due to the second scattering S2, the size of each pore 143 may be 0.5 μm or more in radius. In this regard, the radii of the pores 143 may be based on a longer axis. In more detail, the size of each pore 143 may be 1 μm or more in radius. Also, in this case, the surface roughness of at least one of the first surface 141 and the second surface 142 may be 20 nm or less based on root mean square (rms).

A more detailed embodiment of the light diffuser 14 as described above is as follows.

A coating composition solution is prepared.

According to an embodiment, the coating composition solution may include colorless polyamic acid.

The coating composition solution may be prepared by mixing 4,4′-oxydiphthalic anhydride and 2,2-bis[4-(4-aminophenoxy)phenyl] hexafluoropropane in a dimethylacetamide (DMAc) solvent at a molar ratio of 1:1, stirring the mixture for 24 hours, and then diluting the resultant liquid with a 3 w % DMAc solvent.

Next, the coating composition solution is coated on a third film 13.

The third film 13 coated with the coating composition solution is supported in a pore-forming solvent.

A polar protic solvent may be used as the pore-forming solvent, and the pore-forming solvent may include alcohol.

In Example, 100% of de-ionized water (DIW) was used as the pore-forming solvent. In Comparative Example, 100% of ethanol was used.

The thus formed pore-forming solvents through Example and Comparative Example were subject to heat drying at 170° C., thereby forming a polyimide-based base material 140.

### Mode of Disclosure

(a) of FIG. 4 is a cross-sectional scanning electron microscope (SEM) photograph of an embodiment, and (b) of FIG. 4 is a surface SEM photograph of an embodiment. (a) of FIG. 5 is a cross-sectional SEM photograph of a comparative example, and (b) of FIG. 5 is a surface SEM photograph of a comparative example. In the embodiment shown in FIG. 4, a light extraction structure in which a first scattering degree due to a pore is greater than a second scattering degree due to a surface is provided. In the embodiment shown in FIG. 5, a light extraction structure in which a second scattering degree due to a surface is greater than a first scattering degree due to a pore is provided.

A thickness of a base material that is a formed layer is 3.1 μm in Example and is 1.3 μm in Comparative Example. As such, it may be seen that, with respect to a layer having the same composition, the thickness of the layer of Example is greater than the thickness of the layer of Comparative Example.

For a size of a formed pore, a maximum pore size (based on a longer axis) is about 3 μm in Example and is about 1.3 μm in Comparative Example. It may be seen that the size of the pore in Example is significantly greater than the size of the pore in Comparative Example.

A surface roughness (based on root mean square (rms)) is 3.6 nm in Example and is 68 nm in Comparative Example. It may be seen that the surface roughness of Example is significantly less than the surface roughness of Comparative Example.

As another embodiment, the second film 14 that is the light diffuser may be prepared by using a photocatalyst in a fluoropolymer.

Hexafluoropropylene, vinylidene fluoride, and/or tetrafluoroethylene are mixed at a molar ratio of 1:1.

The mixture is mixed again with an n-butyl acetate solvent at a molar ratio of 1:4 to prepare a 1H,1H,2H,2H-perfluorodecyltriethoxysilane solution.

Then, the mixture is mixed with zirconium dioxide (ZrO₂) at a molar ratio of 1:3 to prepare a final solution.

This solution is coated on the third film 13 and then dried at 150° C. A transmittance and haze may be adjustable according to a thickness of the light diffuser to be coated.

The light diffuser may be formed with a thickness of 40 μm in Example 1, the light diffuser may be formed with a thickness of 200 μm in Example 2, and the light diffuser may be formed with a thickness of 500 μm in Example 3.

In this case, average light transmittances were 76% in Example 1, 51% in Example 2, and 28% in Example 3. In addition, average haze values were 94% in Example 1, 99% in Example 2, and 99% in Example 3.

As can be seen in this experiment, it is desirable to make the thickness of the light diffuser as thin as possible.

As another embodiment, the second film 14 that is the light diffuser may be prepared with an acrylic curable polymer having a molecular weight of 30,000 or more.

The acrylic polymer may include glycidyl methacrylate.

In more detail, acrylic monomers including glycidyl methacrylate are mixed with propylene glycol ether acetate, and a temperature of a reactor is raised to 60° C., followed by stirring the mixture under a nitrogen atmosphere.

Thereafter, when a temperature of the mixture reaches 60° C., the mixture is added with a thermal polymerization initiator, for example, azodiisobutyronitrile (AlBN), and then stirred. The stirring may be performed for 12 hours, for example.

Thereafter, the solution is coated on the third film 13 and then dried.

The light diffuser may be provided with a curable polymer resin having a solid content of about 35% to about 45%, and a molecular weight of 30,000 g/mol or more.

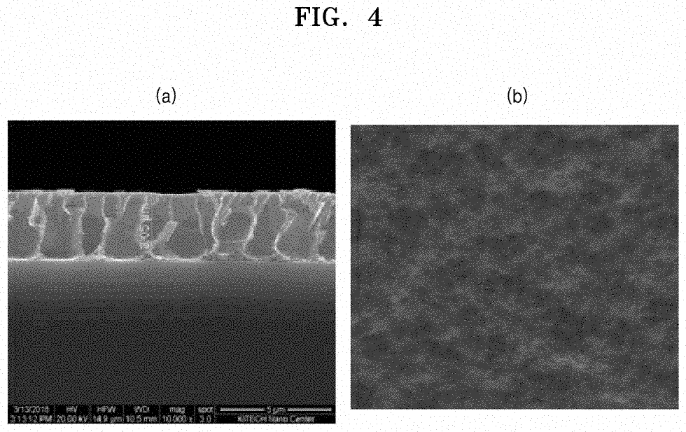

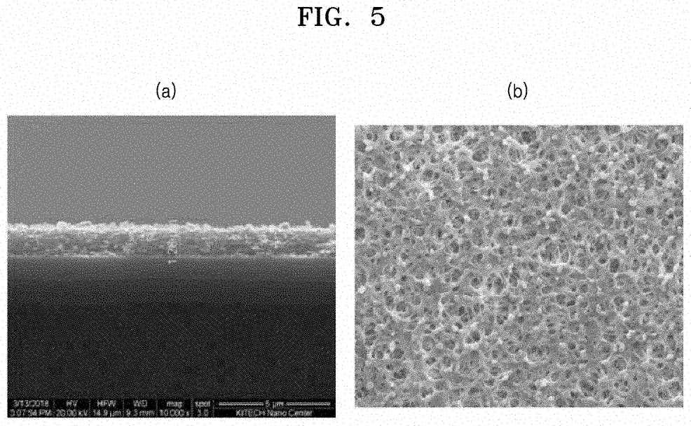

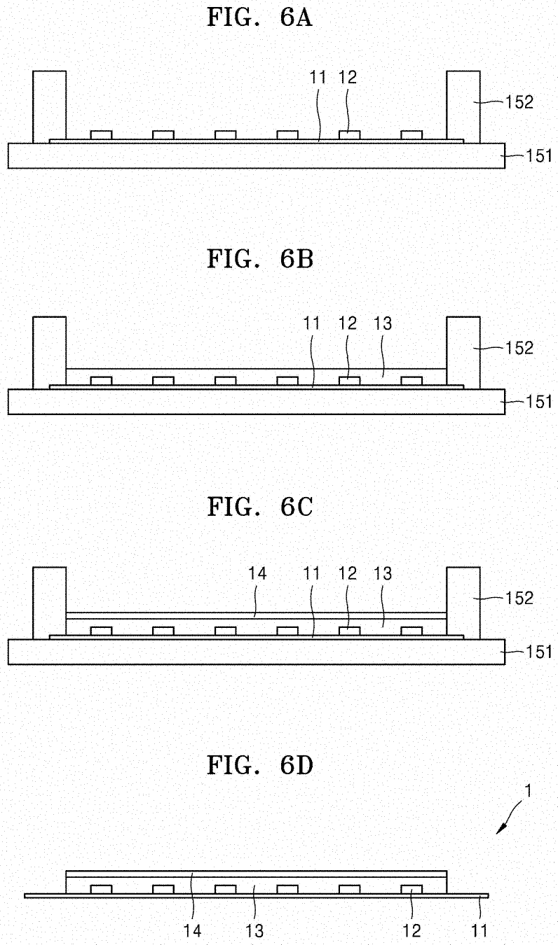

The embodiments as described above may be formed by using a method of FIGS. 6A to 6D.

As shown in FIG. 6A, the flexible substrate 11 including the light-emitting elements 12 is arranged on a supporter 151, and a mold 152 is formed at an edge of the flexible substrate 11.

Next, as shown in FIG. 6B, the third film 13 is formed by applying, into the mold 152, a composition solution capable of forming a third film, and then drying the composition solution. The composition solution may be poured into the mold 152 and uniformly coated by means of blade coating and/or spin coating.

Next, as shown in FIG. 6C, a fourth film 14 is formed by coating, into the mold 152, with a composition solution capable of forming a fourth film, and then drying the composition solution. The composition solution may be poured into the mold 152 and uniformly coated by means of blade coating and/or spin coating.

Next, as shown in FIG. 6D, the flexible light-emitting assembly 1 may be obtained by removing the mold 152.

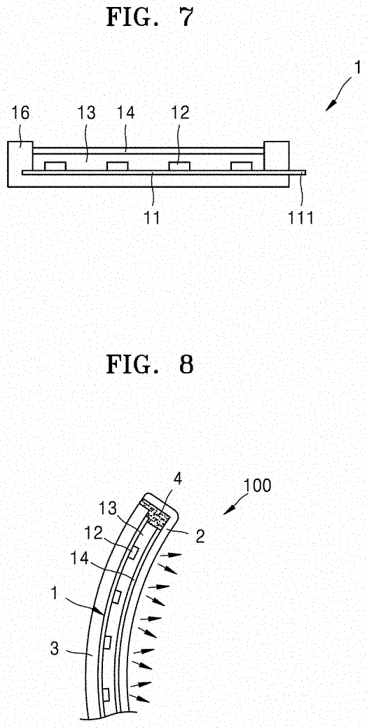

FIG. 7 is a schematic cross-sectional view of a flexible light-emitting assembly 1 according to another embodiment.

As described above, the flexible substrate 11 including the light-emitting elements 12 is prepared, and the first film 13 is formed on the flexible substrate 11. The first film 13 may include a transmissive silicone material.

Next, the second film 14 that is the light diffuser is arranged on the first film 13. The second film 14 may be separately prepared and arranged on the first film 13.

In a state in which the second film 14 is arranged on the first film 13, the flexible substrate 11 is sealed with an envelope 16. The envelope 16 may cover a lower portion of the flexible substrate 11, side surfaces of the first film 13, and side surfaces of the second film 14, so that the flexible substrate 11 is sealed. The envelope 16 may include a transmissive silicone material.

Although not shown in the FIG. 7, according to another embodiment, the envelope 16 may be formed to cover an upper surface of the second film 14, and accordingly, a stack of the flexible substrate 11, the first film 13, and the second film 14 may be entirely surrounded. In this case, because all internal structures are sealed by the envelope 16, the durability of internally sealed components may be further improved.

The envelope 16 may be formed by using an insert injection molding method.

In the above-described embodiments, the second film 14 has been described as the light diffuser, but the disclosure is not limited thereto. The first film 13 may be the light diffuser. In another embodiment, only one of the first film 13 and the second film 14 may be provided, and the one film may be the light diffuser.

The flexible light diffuser as described above may transfer energy of each wavelength of the light-emitting element as it is without any changes in a spectrum from an ultraviolet (UV) light region to a visible light region. That is, light transmitted through the light diffuser may be transferred to an irradiation surface by minimizing loss in the light power. According to an embodiment, the light diffuser does not cause a shift in wavelengths, and a wavelength of the light transmitted may be maintained as it is.

Also, because the light diffuser may form a uniform in-plane light source, light energy may be uniformly transferred over an entire surface.

Moreover, because the light diffuser is flexible, uniform light characteristics do not change even though the light diffuser is applied to uneven skin. Also, due to a diffusing function, a micro element may be used as the light-emitting element, and even when a micro light-emitting element is used, a sufficient in-plane light source can be achieved, so that the number of light-emitting elements may be reduced. When the number of light-emitting elements is reduced as described above, a heat generation problem caused due to a great number of light-emitting elements may be solved.

FIG. 8 is a cross-sectional view of a portion of a light therapy device 100 according to an embodiment.

Referring to FIG. 8, the light therapy device 100 according to an embodiment may include an inner cover unit 2, an outer cover unit 3, and the flexible light-emitting assembly 1.

The inner cover unit 2 may include a portion facing user's skin and may include a transmissive material, which may be a material that less irritates the user's skin.

The inner cover unit 2 may be formed to in a curved shape to correspond to curves of the user's skin and may include a human-friendly material. The inner cover unit 2 may include a silicone material. Accordingly, the inner cover unit 2 may be stably seated on the user's skin without slipping thereon.

Optionally, the inner cover unit 2 may include a polyurethane material. Because the polyurethane material has a certain degree of elasticity, the wearability may be increased when in contact with the skin.

Optionally, the inner cover unit 2 may include an elastomer material. The elastomer material may exhibit rubber elasticity at room temperature. Thus, the elastomer material does not damage the skin when in contact with the skin and may improve the wearability.

The outer cover unit 3 may be positioned to face the inner cover unit 2, and may have a certain space with respect to the inner cover unit 2.

The outer cover unit 3 may be provided with curves in the same manner as the curves of the inner cover unit 2 and may be coupled to the inner cover unit 2 at an edge thereof. The outer cover unit 3 may include a material more rigid than the material of the inner cover unit 2, and accordingly, overall rigidity may be maintained. Also, the outer cover unit 3 may include a non-transmissive material, and accordingly, a problem in which light leaks to the outside may be prevented, and glare or the like may not occur during use.

The flexible light-emitting assembly 1 may be arranged between the inner cover unit 2 and the outer cover unit 3. For example, the light-emitting elements 12 are arranged on the flexible substrate 11, and the first film 13 and the second film 14 may be formed to cover the flexible substrate 11.

In this regard, as described above, the second film 14 is provided as the light diffuser, so that light emitted from the light-emitting elements 12 toward the inner cover unit 2 may be further diffused so as to be uniformly distributed and irradiated over the skin.

The flexible light-emitting assembly 1 is not limited to the embodiment shown in FIG. 8 but may be applied to all the various embodiments described above in association with the flexible light-emitting assembly 1.

A space between the inner cover unit 2 and the outer cover unit 3 assembled with each other with the flexible light-emitting assembly 1 therebetween may be sealed by a sealant 4 which is a molding support. The sealant 4 may include a silicone material and may be inserted into the space between the inner cover unit 2 and the outer cover unit 3 by means of insert injection.

Foreign matter/moisture may not penetrate into the space between the inner cover unit 2 and the outer cover unit 3 by the sealant 4, and thus durability of the flexible light-emitting assembly 1 may be improved.

Optionally, the sealant 4 may serve to prevent light from leaking out through the space between the inner cover unit 2 and the outer cover unit 3. To this end, the sealant 4 may include an opaque material and/or a light absorbing material.

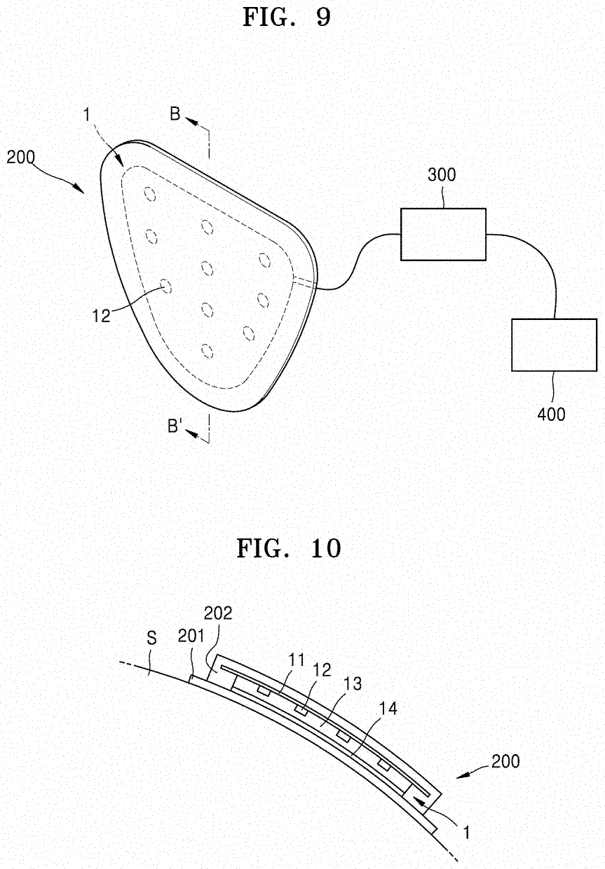

FIG. 9 illustrates a light therapy device 200 according to another embodiment, and the light therapy device 200 according to another embodiment may be provided as a patch-type light therapy device 200 attachable to the skin.

The patch-type light therapy device 200 may include a flexible light-emitting assembly 1 therein, and the flexible light-emitting assembly 1 may include light-emitting elements 12 emitting light toward the skin.

The light-emitting elements 12 may be electrically connected to a power source 400 via a controller 300.

The power source 400 provides electricity to the light-emitting elements 12 and may be a household power source. However, the power source 400 is not limited thereto, and may include a device including an electric capacitor, such as a portable auxiliary battery, a portable terminal, a personal computer device, etc.

The controller 300 may adjust the degree of light emission of the light-emitting elements 12. In an embodiment, the controller 300 may adjust not only turning on/off, but also the luminance in stages. Moreover, the controller 300 may include a dimming switch.

FIG. 10 is a cross-sectional view of a light therapy device taken along a line B-B of FIG. 9.

Referring to FIG. 10, the patch-type light therapy device 200 is arranged to emit light toward skin S, and a flexible light diffuser 14 is further arranged on a light path from the light-emitting elements 12 toward the skin S, so that the light is uniformly diffused over the skin S.

A bonding pad 201, which is a bonding support, may be provided adjacent to the skin S, and a body 202 sealing the flexible light-emitting assembly 1 may be coupled to the bonding pad 201.

The body 202 may have an insert-injection molding structure formed using silicone material with the flexible light-emitting assembly 1 therebetween. The body 202 may include a non-transparent material, and thus a problem in which light leaks to the outside may be prevented.

The flexible light-emitting assembly 1 may be applied to all the various embodiments described above.

The bonding pad 201 may include a transmissive elastic material and may include at least one of silicone, polyurethane, and an elastomer.

Because the patch-type light therapy device 200 may be attached to a desired location on the skin S and thus used, a user may conveniently receive light therapy.

In this regard, due to the afore-mentioned embodiments of the flexible light-emitting assembly 1, a uniform light emission effect may be achieved without loss of light energy while reducing a heat generation problem, thereby enabling safer and more efficient therapy. Also, due to the flexible light-emitting assembly 1, the light therapy device may be attached to an excessively uneven part of skin, which may solve a problem of deteriorating a light therapy effect.

All of the above-described embodiments may be applied in conjunction with each other.

Although the disclosure has been described with reference to the embodiments shown in the accompanying drawings, the embodiments are merely provided as examples. It will be understood by those of ordinary skill in the art that various modifications and other equivalent embodiments may be made therefrom. Accordingly, the true protection scope of the disclosure should be defined by the appended claims below.

### INDUSTRIAL APPLICABILITY

The disclosure is applicable in the field of light therapy devices.

1. A light therapy device comprising:
a first support positioned adjacent to skin, the first support being transmissive;
a second support positioned opposite to the first support, the second support being non-transmissive; and
a flexible light-emitting assembly positioned between the first support and the second support, the flexible light-emitting assembly including a light diffuser and a light-emitting element,
wherein the light diffuser includes a base material including a first surface and a second surface, which face each other, the base material being configured to allow light of the light-emitting element to travel from the first surface to the second surface,
wherein the base material is configured to scatter the light when the light passes therethrough.

2. The light therapy device of claim 1, wherein
the base material includes a plurality of pores irregularly distributed in the base material,
the scattering includes first scattering due to the pores and second scattering due to at least one of the first surface and the second surface, and
a first scattering degree due to the first scattering and a second scattering degree due to the second scattering have a relative difference.

3. The light therapy device of claim 2, wherein,
when the first scattering degree due to the first scattering is greater than the second scattering degree due to the second scattering, each of the pores has a first diameter, and when the second scattering degree due to the second scattering is greater than the first scattering degree due to the first scattering, each of the pores has a second diameter,
wherein the first diameter is greater than the second diameter.

4. The light therapy device of claim 2, wherein,
a roughness of at least one of the first surface and the second surface when the first scattering degree due to the first scattering is greater than the second scattering degree due to the second scattering, is a first roughness, and a roughness of at least one of the first surface and the second surface when the second scattering degree due to the second scattering is greater than the first scattering degree due to the first scattering, is a second roughness,
wherein the first roughness is less than the second roughness.

5. The light therapy device of claim 1, further comprising
a molding support positioned between the first support and the second support.

6. The light therapy device of claim 1, further comprising
a bonding support positioned between the skin and the light diffuser, the bonding support being bondable to the skin.
